(19) United States
(12) Patent Application Publication
(10) Pub. No.: US 2023/0398083 A1
(43) Pub. Date: Dec. 14, 2023

(54) KETOPROFEN-CONTAINING PATCH

(71) Applicant: TEIKOKU SEIYAKU CO., LTD., Kagawa (JP)

(72) Inventor: Katsuyuki INOO, Kita-gun, Kagawa (JP)

(73) Assignee: TEIKOKU SEIYAKU CO., LTD., Kagawa (JP)

(21) Appl. No.: 18/033,235

(22) PCT Filed: Oct. 22, 2021
(86) PCT No.: PCT/JP2021/039140

§ 371 Date: Apr. 21, 2023

(30) Foreign Application Priority Data
Oct. 23, 2020 (JP) 2020-178065

(51) Int. Cl. A61K 9/70; A61K 31/192; A61K 47/02; A61K 47/10; A61K 47/14; A61K 47/32

(52) U.S. Cl. CPC A61K 9/7038; A61K 31/192; A61K 47/02; A61K 47/10; A61K 47/14; A61K 47/32

(57) ABSTRACT

The present invention provides a patch which prevents ketoprofen from forming a menthol ester, suppresses crystal precipitation, and yet shows excellent skin permeability. Specifically, the present invention provides a patch comprising ketoprofen, L-menthol, zinc oxide, and a fatty acid ester in a pasty preparation.

### TECHNICAL FIELD

The present invention relates to a patch comprising ketoprofen, L-menthol, zinc oxide, and a fatty acid ester.

### BACKGROUND ART

Ketoprofen is one of the nonsteroidal anti-inflammatory analgesics which exhibits excellent anti-inflammatory analgesic effects, and has also been used as an external agent for a long time. In patches, L-menthol is often blended as a refreshing agent or the like, but nonsteroidal anti-inflammatory analgesics having a carboxylic acid group in molecules such as ketoprofen react with menthol to produce menthol esters, and it has long been known that problems occur in the stability of the drug. Regarding the stability problem due to the formation of menthol esters of nonsteroidal anti-inflammatory analgesics having a carboxylic acid group in the molecule, a technique for improving stability by adding metal compounds such as metal oxides, metal hydroxides, and metal carbonates has already been disclosed (Patent Documents 1 to 3). However, since it has been confirmed that the addition of the above metal compounds suppresses drug release from the patches, further improvement is desired.

### CITATION LIST

### Patent Document

Patent Document 1: JP 2002-226366 A
Patent Document 2: JP 2005-314328 A
Patent Document 3: JP 2010-90098 A

### SUMMARY OF INVENTION

### Technical Problem

An object of the present invention is to provide patches which suppress the production of menthol esters of ketoprofen, suppress crystal precipitation, and yet show excellent skin permeability.

### Solution to Problem

The present inventor has earnestly studied in order to solve the above problems. As a result, he has found that when a patch comprising ketoprofen and L-menthol also comprises zinc oxide as well as a fatty acid ester, such patch can suppress the production of a menthol ester of ketoprofen and yet shows excellent skin permeability, and finally completed the present invention. Surprisingly, the present inventor has also found that the present invention can also suppress crystal precipitation of ketoprofen with time.

The present invention provides an external patch characterized by comprising ketoprofen, L-menthol, zinc oxide, and a fatty acid ester.

Namely, the present invention relates to the followings.

[1] A patch comprising ketoprofen, L-menthol, zinc oxide, and a fatty acid ester in a pasty preparation (hereinafter also referred to as “Present patch”).
[2] The patch according to [1], wherein the fatty acid ester is one or more selected from isopropyl myristate, isopropyl palmitate, isopropyl isostearate, diisopropyl adipate, and diethyl sebacate.
[3] The patch according to [1] or [2], wherein the fatty acid ester is one or two selected from isopropyl myristate and diisopropyl adipate.
[4] The patch according to any one of [1] to [3], wherein the fatty acid ester is a combination of a fatty acid diester and a fatty acid monoester.
[5] The patch according to any one of [1] to [4], wherein the contained amount of the ketoprofen is 0.1 to 10% by weight, the contained amount of the L-menthol is 0.1 to 10% by weight, the contained amount of the zinc oxide is 0.03 to 0.5% by weight, and the contained amount of the fatty acid ester is 0.1 to 20% by weight, relative to the pasty preparation weight.
[6] The patch according to any one of [1] to [5], wherein the weight ratio of the contained amount of the ketoprofen and the contained amount of the zinc oxide is 1:0.03 to 1:0.1.
[7] The patch according to any one of [1] to [6] further comprising one or more ingredient(s) selected from a base polymer, a tackifier resin, and a softener in the pasty preparation.
[8] The patch according to [7], wherein
the base polymer is a styrene-isoprene-styrene block copolymer;
the tackifier resin is one or more selected from a hydrogenated rosin glycerol ester, an alicyclic saturated hydrocarbon resin, and a terpene resin; and
the softener is one or two selected from polybutene and liquid paraffin.
   [9] The patch according to [7] or [8], wherein the tackifier resin is one or two selected from a hydrogenated rosin glycerol ester and an alicyclic saturated hydrocarbon resin.
   [10] The patch according to any one of [7] to [9], wherein the contained amount of the base polymer is 5 to 50% by weight, the contained amount of the tackifier resin is 10 to 60% by weight, and the contained amount of the softener is 10 to 60% by weight, relative to the pasty preparation weight.
   [11] The patch according to any one of [1] to further comprising an antioxidant in the pasty preparation.
   [12] The patch according to [11], wherein the antioxidant is dibutylhydroxytoluene.
   [13] The patch according to or [12], wherein the contained amount of the antioxidant is 0.05 to 5% by weight relative to the pasty preparation weight.
   [14] The patch according to any one of [1] to for use in the prevention or treatment of inflammation or pain.

### Effect of Invention

According to the present invention, a patch which suppresses the production of a menthol ester of ketoprofen, suppresses crystal precipitation, and yet shows excellent skin permeability can be provided.

### DESCRIPTION OF EMBODIMENTS

In the present description, “contain” or “comprise” may be used interchangeably with “blend”. Also, in the present description, “contained amount” may be used interchangeably with “amount”.

The Present patch comprises ketoprofen, L-menthol, zinc oxide, and a fatty acid ester in a pasty preparation. The pasty preparation is a pasty composition usually comprising an adhesive base comprising an adhesive as a main ingredient, and a drug, and may also be referred to as “adhesive composition”.

The amount of the ketoprofen contained in the Present patch as an active ingredient is not specifically limited as long as formulating can be carried out, but is usually within a range of 0.1 to 10% by weight, preferably 0.5 to 8% by weight, and more preferably 1 to 5% by weight, relative to the pasty preparation weight. When the contained amount of the ketoprofen in a pasty preparation is less than 0.1% by weight, transdermal absorbability becomes insufficient. Meanwhile, when said amount is more than 10% by weight, the physical properties of the patch are impaired and said amount is also economically disadvantageous.

L-menthol contained in the Present patch functions as a transdermal absorption enhancer and a refreshing agent.

The amount of the L-menthol contained in the Present patch is not specifically limited as long as formulating can be carried out, but is usually within a range of 0.1 to 10% by weight, preferably 0.5 to 8% by weight, and more preferably 1 to 5% by weight, relative to the pasty preparation weight. When the contained amount of the L-menthol in a pasty preparation is less than 0.1% by weight, transdermal absorbability becomes insufficient. Meanwhile, when said amount is more than 10% by weight, the physical properties of the patch are impaired.

Zinc oxide contained in the Present patch has a function for suppressing esterification reaction and crystallization of ketoprofen.

The amount of the zinc oxide contained in the Present patch is usually within a range of 0.03 to 0.5% by weight, preferably 0.06 to 0.3% by weight, and more preferably 0.1 to 0.2% by weight, relative to the pasty preparation weight.

The amount of the zinc oxide contained in the Present patch is preferably varied depending on the contained amount of the ketoprofen in order to suppress the esterification reaction and crystallization of the ketoprofen. When the contained amount of the ketoprofen is set to be “1”, the weight ratio of the contained amount of the zinc oxide is preferably within a range of 0.03 to 0.1, more preferably within a range of 0.04 to 0.1, and still more preferably within a range of 0.05 to 0.1. When the weight ratio of the contained amount of the zinc oxide relative to the ketoprofen is less than 0.03, the effect for suppressing crystal precipitation and esterification reaction becomes insufficient. Meanwhile, when said weight ratio is more than 0.1, skin permeability of the drug is suppressed.

Also, the zinc oxide is not dissolved, but dispersed in the formulation. The zinc oxide contained in the Present patch preferably has a particle size of 1 to 100 nm in view of dispersibility at the time of manufacture.

The fatty acid ester contained in the Present patch has a function for enhancing the dispersibility of the zinc oxide at the time of manufacture and a function for suppressing the decrease of drug permeability due to the presence of the zinc oxide.

The fatty acid ester used in the Present patch is preferably a fatty acid ester which is liquid at ordinary temperature. Further, the fatty acid ester preferably has appropriate viscosity when the zinc oxide is mixed in view of the dispersibility of the zinc oxide. The viscosity of the fatty acid ester to be used is preferably 100 mPa·s or less, and more preferably 5 to 50 mPa·s.

Examples of the fatty acid ester which can be used in the Present patch include hexyl laurate, isopropyl myristate, methyl myristate, myristyl myristate, cetyl myristate, octyldodecyl myristate, isopropyl palmitate, cetyl palmitate, isopropyl isostearate, diisobutyl adipate, diisopropyl adipate, dioctyl adipate, diisopropyl sebacate, diethyl sebacate, ethyl oleate, decyl oleate, oleyl oleate, ethyl linoleate, isopropyl linoleate, cetyl lactate, and ethyl lactate, and one of them or a combination of two or more of them may be used. Preferable examples thereof include one or more selected from isopropyl myristate, isopropyl palmitate, isopropyl isostearate, diisopropyl adipate, and diethyl sebacate, and more preferable examples thereof include one or two selected from isopropyl myristate and diisopropyl adipate.

Also, when two or more fatty acid esters are used in combination, a combination of a fatty acid diester (for example, diisobutyl adipate, diisopropyl adipate, dioctyl adipate, diisopropyl sebacate, and diethyl sebacate) and a fatty acid monoester (for example, hexyl laurate, isopropyl myristate, methyl myristate, myristyl myristate, cetyl myristate, octyldodecyl myristate, isopropyl palmitate, cetyl palmitate, isopropyl isostearate, ethyl oleate, decyl oleate, oleyl oleate, ethyl linoleate, isopropyl linoleate, cetyl lactate, and ethyl lactate) is preferably used.

The amount of the fatty acid ester contained in the Present patch is usually within a range of 0.1 to 20% by weight, preferably 0.5 to 15% by weight, and more preferably 1 to 10% by weight, relative to the pasty preparation weight. When the contained amount of the fatty acid ester in a pasty preparation is less than 0.1% by weight, the effect for enhancing the dispersibility of the zinc oxide and the effect for enhancing the permeation of the drug become insufficient. Meanwhile, when said amount is more than 20% by weight, the physical properties of the formulation are deteriorated.

In one embodiment, the pasty preparation of the Present patch may further comprise one or more ingredient(s) selected from a base polymer, a tackifier resin, and a softener, and may further optionally comprise other ingredient(s).

Examples of the base polymer which can be used in the Present patch include acrylic adhesives, rubber adhesives, and silicone adhesives, and a rubber adhesive is preferably used. Examples of the rubber adhesive include a natural rubber, polyisobutylene, polyisoprene, polybutadiene, a styrene-isoprene-styrene block copolymer (hereinafter also referred to as “SIS”), a styrene-butadiene rubber, and a styrene-isoprene rubber. One of them or a combination of two or more of them may be used, and a styrene-isoprene-styrene block copolymer (SIS) is preferably used.

The amount of the base polymer contained in the Present patch is determined in view of the contained amounts of other ingredients, and usually within a range of 5 to 50% by weight, preferably 10 to 40% by weight, and more preferably 10 to 30% by weight, relative to the pasty preparation weight. When the contained amount of the base polymer in a pasty preparation is less than 5% by weight, the cohesive force and/or shape retention property of the pasty preparation decrease(s). Meanwhile, when the amount is more than 50% by weight, the adhesive force decreases, the pasty preparation become heterogeneous, and the workability during the manufacture decreases.

The tackifier resin is not specifically limited as long as it is generally contained in a patch, and examples thereof include a rosin resin such as a rosin ester and a hydrogenated rosin glycerol ester; a petroleum resin such as an alicyclic saturated hydrocarbon resin; and a terpene resin, and one of them or a combination of two or more of them may be used. Preferable examples thereof include one or more selected from a rosin resin, a petroleum resin, and a terpene resin, such as one or more selected from a hydrogenated rosin glycerol ester, an alicyclic saturated hydrocarbon resin, and a terpene resin, more preferable examples thereof include one or more selected from a rosin resin and a petroleum resin, and still more preferable examples thereof include one or two selected from a hydrogenated rosin glycerol ester and an alicyclic saturated hydrocarbon resin.

The amount of the tackifier resin contained in the Present patch is not specifically limited as long as formulating can be carried out, but is usually within a range of 10 to 60% by weight, preferably 15 to 50% by weight, and more preferably 20 to 40% by weight, relative to the pasty preparation weight. When the contained amount of the tackifier resin in a pasty preparation is less than 10% by weight, the physical properties of the formulation are deteriorated and undesirable phenomena such as adhesive residues occur. Meanwhile, when said amount is more than 60% by weight, skin irritation occurs.

The softener used in the Present patch is not specifically limited as long as it is compatible with the other base ingredients and provides the pasty preparation with plasticity. Examples of the softener which can be used in the Present patch include polyisobutylene, liquid polyisoprene, polybutene, lanolin, castor oil, almond oil, olive oil, camellia oil, persic oil, peanut oil, process oil, extender oil, and liquid paraffin, and one of them or a combination of two or more of them may be used. Preferable examples thereof include one or two selected from polybutene and liquid paraffin.

The amount of the softener contained in the Present patch is determined also in view of the contained amounts of the other liquid ingredients contained in the pasty preparation, and usually within a range of 10 to 60% by weight, preferably 20 to 50% by weight, and more preferably to 45% by weight, relative to the pasty preparation weight.

Also, the Present patch may comprise various base ingredients used in normal patches as long as they do not affect the other ingredients. Examples of such base ingredients include, but are not specifically limited to, water-soluble polymers such as polyvinylpyrrolidone and polyvinyl alcohol; cellulose derivatives such as hydroxypropyl methylcellulose; silicon compounds such as anhydrous silicic acid and light anhydrous silicic acid; antioxidants such as dibutylhydroxytoluene (also referred to as “BHT”), pentaerythrityl-tetrakis-[3-(3,5-di-t-butyl-4-hydroxyphenyl)propionate], tocopherol acetate, and ascorbic acid; and inorganic fillers such as silica and zinc stearate. The Present patch may further comprise preservatives such as p-hydroxybenzoates (for example, methyl p-hydroxybenzoate); fungicides such as ethanol and isopropyl alcohol; flavoring agents such as mentha oil; colorants such as yellow ferric oxide, and the like, if necessary, and they may be optionally contained in the Present patch at appropriate amounts.

In one embodiment, the pasty preparation of the Present patch further comprises an antioxidant. Preferable examples of the antioxidant include dibutylhydroxytoluene.

The amount of the antioxidant contained in the Present patch is usually within a range of 0.05 to 5% by weight, preferably 0.1 to 1% by weight, and more preferably 0.2 to by weight, relative to the pasty preparation weight.

The Present patch usually consists of a backing, a pasty preparation, and a release liner, and the pasty preparation is spread or applied between a backing and a release liner.

Examples of the backing used in the Present patch include films, non-woven fabrics, woven fabrics, knitted fabrics, and laminate composites of non-woven fabrics and films. Examples of the material of these backings include polyethylene, polypropylene, polyvinyl chloride, polyester, polyethylene terephthalate, nylon, polyurethane, rayon, polyacrylonitrile, polystyrene, and polyethylene naphthalate.

Examples of the release liner used in the Present patch include polyethylene terephthalate, polypropylene, and paper, and especially preferable examples thereof include polyethylene terephthalate. The release liner may be siliconized if necessary in order to optimize the release force.

Examples of the production method of the Present patch include, but are not limited to, a solvent method characterized in that an adhesive composition is dissolved into an organic solvent such as toluene and hexane, and the organic solvent is removed by coating and drying steps; and a hot melt method characterized in that an adhesive composition is melted at a high temperature of 100° C. or higher, and then a coating step is carried out. In one embodiment, the Present patch is produced by a hot melt method.

In one embodiment, the method for producing the Present patch comprises:
a step of mixing ketoprofen, zinc oxide, and a fatty acid ester to obtain a drug solution; and
a step of mixing the drug solution, L-menthol, and optionally other ingredient(s) to obtain an adhesive composition.

In another embodiment, the method for producing the Present patch comprises:
a step of heating and dissolving a base polymer, a tackifier resin, a softener, and an antioxidant to obtain an adhesive solution;
a step of mixing ketoprofen, zinc oxide, and a fatty acid ester to obtain a drug solution;
a step of adding L-menthol and the drug solution to the adhesive solution, and mixing the mixture to obtain an adhesive composition;
a step of coating the adhesive composition on a release liner to form an adhesive layer; and
a step of laminating the adhesive layer on a backing.

Because the Present patch comprises ketoprofen as an active ingredient, it is useful in the prevention or treatment of disease(s) or symptom(s) whose clinical condition(s) is/are expected to be improved by administering ketoprofen. Examples of such disease(s) or symptom(s) include inflammation and pain. Also, examples of the inflammation or pain include low back pain (for example, myofascial low back pain, spondylosis deformans, discopathy, and back strain), osteoarthritis, shoulder periarthritis, tendinitis and tenosynovitis, peritendinitis, humeral epicondylitis (for example, tennis elbow), muscle pain, posttraumatic swelling and pain, and joint local pain due to rheumatoid arthritis.

In the present invention, the term of “prevention” means administering the Present patch to an individual who has not developed disease(s) or symptom(s). Also, the term of “treatment” means administering the Present patch to an individual who has already developed disease(s) or symptom(s). Thus, administering the Present patch to an individual who has already developed disease(s) or symptom(s) for the prevention of deterioration, attack, or recurrence of symptom(s) and the like is an aspect of “treatment”.

The Present patch is usually applied to a patient, for example a human or an animal, and preferably a human who suffers from or is at risk of suffering from the above disease(s) or symptom(s). The frequency of application may appropriately vary depending on conditions such as severity of the disease(s) or symptom(s), age, weight, and sex of the patient, and the amount of ketoprofen in patch. When applied to a human, the Present patch is usually exchanged one or more time(s) per day such as 1 to 3 time(s), 1 to 2 time(s), and once per day, or every several days such as every 2 to 3 days.

Hereinafter, aspects of the present invention are described. Aspects produced by optionally combining the following aspects and aspects produced by optionally combining the following aspect(s) with any of aspect(s) or embodiment(s) etc. disclosed in the present description are also encompassed by the present invention.

### 1. Patch

### [Aspect 1]

The Present patch, wherein the fatty acid ester is one or more selected from hexyl laurate, isopropyl myristate, methyl myristate, myristyl myristate, cetyl myristate, octyldodecyl myristate, isopropyl palmitate, cetyl palmitate, isopropyl isostearate, diisobutyl adipate, diisopropyl adipate, dioctyl adipate, diisopropyl sebacate, diethyl sebacate, ethyl oleate, decyl oleate, oleyl oleate, ethyl linoleate, isopropyl linoleate, cetyl lactate, and ethyl lactate.

### [Aspect 2]

The Present patch, wherein the fatty acid ester is one or more selected from isopropyl myristate, isopropyl palmitate, isopropyl isostearate, diisopropyl adipate, and diethyl sebacate.

### [Aspect 3]

The Present patch, wherein the fatty acid ester is one or two selected from isopropyl myristate and diisopropyl adipate.

### [Aspect 4]

The Present patch according to any one of the Aspects 1 to 3, wherein the fatty acid ester is a combination of a fatty acid diester and a fatty acid monoester.

### [Aspect 5]

The Present patch according to any one of the Aspects 1 to 4, wherein the contained amount of the ketoprofen is to 10% by weight, the contained amount of the L-menthol is 0.1 to 10% by weight, the contained amount of the zinc oxide is 0.03 to 0.5% by weight, and the contained amount of the fatty acid ester is 0.1 to 20% by weight, relative to the pasty preparation weight.

### [Aspect 6]

The Present patch according to any one of the Aspects 1 to 4, wherein the contained amount of the ketoprofen is 0.5 to 8% by weight, the contained amount of the L-menthol is 0.5 to 8% by weight, the contained amount of the zinc oxide is 0.06 to 0.3% by weight, and the contained amount of the fatty acid ester is 0.5 to 15% by weight, relative to the pasty preparation weight.

### [Aspect 7]

The Present patch according to any one of the Aspects 1 to 4, wherein the contained amount of the ketoprofen is 1 to 5% by weight, the contained amount of the L-menthol is 1 to 5% by weight, the contained amount of the zinc oxide is 0.1 to 0.2% by weight, and the contained amount of the fatty acid ester is 1 to 10% by weight, relative to the pasty preparation weight.

### [Aspect 8]

The Present patch according to any one of the Aspects 1 to 7, wherein the weight ratio of the contained amount of the ketoprofen and the contained amount of the zinc oxide is 1:0.03 to 1:0.1.

### [Aspect 9-1]

The Present patch, wherein the contained amount of the ketoprofen is 0.1 to 10% by weight, the contained amount of the L-menthol is 0.1 to 10% by weight, the contained amount of the zinc oxide is 0.03 to 0.5% by weight, and the contained amount of the fatty acid ester is 0.1 to 20% by weight, relative to the pasty preparation weight;
the fatty acid ester is one or more selected from isopropyl myristate, isopropyl palmitate, isopropyl isostearate, diisopropyl adipate, and diethyl sebacate; and
the weight ratio of the contained amount of the ketoprofen and the contained amount of the zinc oxide is 1:0.03 to 1:0.1.

### [Aspect 9]

The Present patch, wherein the contained amount of the ketoprofen is 0.1 to 10% by weight, the contained amount of the L-menthol is 0.1 to 10% by weight, the contained amount of the zinc oxide is 0.03 to 0.5% by weight, and the contained amount of the fatty acid ester is 0.1 to 20% by weight, relative to the pasty preparation weight;
the fatty acid ester is one or two selected from isopropyl myristate and diisopropyl adipate; and
the weight ratio of the contained amount of the ketoprofen and the contained amount of the zinc oxide is 1:0.03 to 1:0.1.

### [Aspect 10]

The Present patch according to any one of the Aspects 1 to 9, wherein the weight ratio of the contained amount of the ketoprofen and the contained amount of the zinc oxide is 1:0.04 to 1:0.1.

### [Aspect 11]

The Present patch according to any one of the Aspects 1 to 9, wherein the weight ratio of the contained amount of the ketoprofen and the contained amount of the zinc oxide is 1:0.05 to 1:0.1.

### [Aspect 12]

The Present patch according to any one of the Aspects 1 to 11, wherein the particle size of the zinc oxide is 1 to 100 nm.

### [Aspect 13]

The Present patch according to any one of the Aspects 1 to 12, wherein the viscosity of the fatty acid ester is 100 mPa·s or less.

### [Aspect 14]

The Present patch according to any one of the Aspects 1 to 12, wherein the viscosity of the fatty acid ester is 5 to 50 mPa·s.

### [Aspect 15]

The Present patch according to any one of the Aspects 1 to 14 further comprising one or more ingredient(s) selected from a base polymer, a tackifier resin, and a softener in the pasty preparation.

### [Aspect 16]

The Present patch according to the Aspect 15, wherein the base polymer is a rubber adhesive.

### [Aspect 17]

The Present patch according to the Aspect 16, wherein the rubber adhesive is one or more selected from a natural rubber, polyisobutylene, polyisoprene, polybutadiene, a styrene-isoprene-styrene block copolymer, a styrene-butadiene rubber, and a styrene-isoprene rubber.

### [Aspect 18]

The Present patch according to the Aspect 16, wherein the rubber adhesive is a styrene-isoprene-styrene block copolymer.

### [Aspect 19-1]

The Present patch according to any one of the Aspects to 18, wherein the tackifier resin is one or more selected from a rosin resin, a petroleum resin, and a terpene resin.

### [Aspect 19]

The Present patch according to any one of the Aspects to 18, wherein the tackifier resin is one or more selected from a rosin resin and a petroleum resin.

### [Aspect 20-1]

The Present patch according to any one of the Aspects to 18, wherein the tackifier resin is one or more selected from a hydrogenated rosin glycerol ester, an alicyclic saturated hydrocarbon resin, and a terpene resin.

### [Aspect 20]

The Present patch according to any one of the Aspects 15 to 18, wherein the tackifier resin is one or two selected from a hydrogenated rosin glycerol ester and an alicyclic saturated hydrocarbon resin.

### [Aspect 21]

The Present patch according to any one of the Aspects 15 to 20, wherein the softener is one or more selected from polyisobutylene, liquid polyisoprene, polybutene, lanolin, castor oil, almond oil, olive oil, camellia oil, persic oil, peanut oil, process oil, extender oil, and liquid paraffin.

### [Aspect 22]

The Present patch according to any one of the Aspects 15 to 20, wherein the softener is one or two selected from polybutene and liquid paraffin.

### [Aspect 23-1]

The Present patch according to the Aspect 15, wherein
the base polymer is a styrene-isoprene-styrene block copolymer;
the tackifier resin is one or more selected from a hydrogenated rosin glycerol ester, an alicyclic saturated hydrocarbon resin, and a terpene resin; and
the softener is one or two selected from polybutene and liquid paraffin.

### [Aspect 23]

The Present patch according to the Aspect 15, wherein
the base polymer is a styrene-isoprene-styrene block copolymer;
the tackifier resin is one or two selected from a hydrogenated rosin glycerol ester and an alicyclic saturated hydrocarbon resin; and
the softener is one or two selected from polybutene and liquid paraffin.

### [Aspect 24]

The Present patch according to any one of the Aspects to 23, wherein the contained amount of the base polymer is 5 to 50% by weight, the contained amount of the tackifier resin is 10 to 60% by weight, and the contained amount of the softener is 10 to 60% by weight, relative to the pasty preparation weight.

### [Aspect 25]

The Present patch according to any one of the Aspects to 23, wherein the contained amount of the base polymer is 10 to 40% by weight, the contained amount of the tackifier resin is 15 to 50% by weight, and the contained amount of the softener is 20 to 50% by weight, relative to the pasty preparation weight.

### [Aspect 26]

The Present patch according to any one of the Aspects 15 to 23, wherein the contained amount of the base polymer is 10 to 30% by weight, the contained amount of the tackifier resin is 20 to 40% by weight, and the contained amount of the softener is 30 to 45% by weight, relative to the pasty preparation weight.

### [Aspect 27]

The Present patch according to any one of the Aspects 1 to 26 further comprising an antioxidant in the pasty preparation.

### [Aspect 28]

The Present patch according to the Aspect 27, wherein the antioxidant is one or more selected from dibutylhydroxytoluene, pentaerythrityl-tetrakis-[3-(3,5-di-t-butyl-4-hydroxyphenyl)propionate], tocopherol acetate, and ascorbic acid.

### [Aspect 29]

The Present patch according to the Aspect 27, wherein the antioxidant is dibutylhydroxytoluene.

### [Aspect 30]

The Present patch according to any one of the Aspects 27 to 29, wherein the contained amount of the antioxidant is 0.05 to 5% by weight relative to the pasty preparation weight.

### [Aspect 31]

The Present patch according to any one of the Aspects 27 to 29, wherein the contained amount of the antioxidant is 0.1 to 1% by weight relative to the pasty preparation weight.

### [Aspect 32]

The Present patch according to any one of the Aspects 27 to 29, wherein the contained amount of the antioxidant is 0.2 to 0.5% by weight relative to the pasty preparation weight.

### [Aspect 33]

The Present patch according to any one of the Aspects 1 to 32 further comprising one or more ingredient(s) selected from a water-soluble polymer, a cellulose derivative, a silicon compound, an inorganic filler, a preservative, a fungicide, a flavoring agent, and a colorant in a pasty preparation.

### [Aspect 34]

The Present patch according to the Aspect 33, wherein
the water-soluble polymer is one or two selected from polyvinylpyrrolidone and polyvinyl alcohol;
the cellulose derivative is hydroxypropyl methylcellulose;
the silicon compound is one or two selected from anhydrous silicic acid and light anhydrous silicic acid;
the inorganic filler is one or more selected from silica and zinc stearate;
the preservative is p-hydroxybenzoate;
the fungicide is one or two selected from ethanol and isopropyl alcohol;
the flavoring agent is mentha oil; and
the colorant is yellow ferric oxide.

### [Aspect 35-1]

The Present patch comprising:
0.1 to 10% by weight of ketoprofen;
0.1 to 10% by weight of L-menthol;
0.03 to 0.5% by weight of zinc oxide;
0.1 to 20% by weight of one or more fatty acid ester(s) selected from isopropyl myristate, isopropyl palmitate, isopropyl isostearate, diisopropyl adipate, and diethyl sebacate;
5 to 50% by weight of a styrene-isoprene-styrene block copolymer;
10 to 60% by weight of one or more tackifier resin(s) selected from a hydrogenated rosin glycerol ester, an alicyclic saturated hydrocarbon resin, and a terpene resin;
10 to 60% by weight of one or two softener(s) selected from polybutene and liquid paraffin; and
0.05 to 5% by weight of dibutylhydroxytoluene relative to the pasty preparation weight;
wherein the weight ratio of the contained amount of the ketoprofen and the contained amount of the zinc oxide is 1:0.03 to 1:0.1.

### [Aspect 35]

The Present patch comprising:
0.1 to 10% by weight of ketoprofen;
0.1 to 10% by weight of L-menthol;
0.03 to 0.5% by weight of zinc oxide;
0.1 to 20% by weight of one or two fatty acid ester(s) selected from isopropyl myristate and diisopropyl adipate;
5 to 50% by weight of a styrene-isoprene-styrene block copolymer;
10 to 60% by weight of one or two tackifier resin(s) selected from a hydrogenated rosin glycerol ester and an alicyclic saturated hydrocarbon resin;
10 to 60% by weight of one or two softener(s) selected from polybutene and liquid paraffin; and
0.02 to 5% by weight of dibutylhydroxytoluene relative to the pasty preparation weight;
wherein the weight ratio of the contained amount of the ketoprofen and the contained amount of the zinc oxide is 1:0.03 to 1:0.1.

### 2. Production Method and Use of Patch

### [Aspect 36]

A method for producing the Present patch according to any one of the Aspects 1 to 35, which comprises:
a step of mixing the ketoprofen, the zinc oxide, and the fatty acid ester to obtain a drug solution; and
a step of mixing the drug solution, the L-menthol, and optionally other ingredient(s) to obtain an adhesive composition.

### [Aspect 37]

The Present patch according to any one of the Aspects 1 to 35 for use in the prevention or treatment of inflammation or pain.

### [Aspect 38]

Use of the Present patch according to any one of the Aspects 1 to 35 in the manufacture of a medicament for preventing or treating inflammation or pain.

### [Aspect 39]

A method for preventing or treating inflammation or pain, which comprises administering the Present patch according to any one of the Aspects 1 to 35 to a patient.

### [Aspect 40]

Use of the Present patch according to any one of the Aspects 1 to 35 in the prevention or treatment of inflammation or pain.

### EXAMPLES

Hereinafter, the present invention is more specifically illustrated by means of Examples, but the present invention is not limited to the following Examples.

In the Examples (Ex.) and Comparative Examples (Com.), numerical values are expressed in “% by weight”, unless otherwise specified.

### Example 1

Each ingredient of a SIS (SIS5002 manufactured by JSR Corporation), an alicyclic saturated hydrocarbon resin (ARKON P100 manufactured by Arakawa Chemical Industries, Ltd.), polybutene (HV-300F manufactured by JXTG Nippon Oil & Energy Corporation), liquid paraffin (HICALL M-352 manufactured by KANEDA Co., Ltd.), and BHT was stirred and dissolved under heating under nitrogen atmosphere to obtain an adhesive solution. Ketoprofen, zinc oxide, diisopropyl adipate, and isopropyl myristate were mixed to obtain a drug solution. To the adhesive solution were added L-menthol and the drug solution, and the resulting mixture was further stirred and mixed to obtain an adhesive composition. The resulting adhesive composition was coated on a siliconized polyethylene terephthalate film to form an adhesive layer having a thickness of about 150 μm. The resulting adhesive layer was laminated on a polyester woven fabric as a backing to obtain a Present patch. The contained amount of each ingredient is shown in Table 1.

### Examples 2 to 9

Each patch of Examples 2 to 9 comprising the ingredients shown in Tables 1 to 3 was obtained according to the production method described in the Example 1.

### Examples 10 to 18

Each patch of Examples 10 to 18 comprising the ingredients shown in Tables 5 to 7 was obtained according to the production method described in the Example 1. In this regard, PX1150N (manufactured by Yasuhara Chemical Co., Ltd.) was used as the terpene resin in Examples 10 and 11, D1161JS (manufactured by Kraton Polymers Japan Ltd.) was used as the SIS in Examples 11 and 12, ARKON P115 (manufactured by Arakawa Chemical Industries, Ltd.) was used as the alicyclic saturated hydrocarbon resin in Example 13, and the other ingredients were the same as those used in the Example 1.

### Comparative Examples 1, 2, 4, and 5

Each patch of Comparative Examples 1, 2, 4, and 5 comprising the ingredients shown in Table 3 or 4 was obtained according to the production method described in the Example 1.

### Comparative Example 3

Each ingredient of a SIS (SIS5002 manufactured by JSR Corporation), an alicyclic saturated hydrocarbon resin (ARKON P100 manufactured by Arakawa Chemical Industries, Ltd.), polybutene (HV-300F manufactured by JXTG Nippon Oil & Energy Corporation), a two-thirds amount of liquid paraffin (HICALL M-352 manufactured by KANEDA Co., Ltd.), and BHT was stirred and dissolved under heating under nitrogen atmosphere to obtain an adhesive solution. Ketoprofen, zinc oxide, and a one-third amount of liquid paraffin were mixed to obtain a drug solution. To the adhesive solution were added L-menthol and the drug solution, and the resulting mixture was further stirred and mixed to obtain an adhesive composition. The resulting adhesive composition was coated on a siliconized polyethylene terephthalate film to form an adhesive layer having a thickness of about 150 μm. The resulting adhesive layer was laminated on a polyester woven fabric as a backing to obtain a patch of Comparative Example 3. The contained amount of each ingredient is shown in Table 3.

TABLE 1

| | Ex. 1 | Ex. 2 | Ex. 3 | Ex. 4 |
|---|---|---|---|---|
| SIS | 20 | 20 | 20 | 20 |
| Alicyclic saturated | 37 | 37 | 37 | 37 |
| hydrocarbon resin | | | | |
| Polybutene | 5 | 5 | 5 | 5 |
| Liquid paraffin | 26.1 | 26.08 | 26.07 | 26.06 |
| BHT | 0.3 | 0.3 | 0.3 | 0.3 |
| Ketoprofen | 2 | 2 | 2 | 2 |
| L-menthol | 2.5 | 2.5 | 2.5 | 2.5 |
| Zinc oxide | 0.1 | 0.12 | 0.13 | 0.14 |
| Diisopropyl adipate | 5 | 5 | 5 | 5 |
| Isopropyl myristate | 2 | 2 | 2 | 2 |
| Ketoprofen:Zinc oxide | 1:0.05 | 1:0.06 | 1:0.065 | 1:0.07 |
| Cumulative permeation | 116.0 | 113.3 | 107.5 | 92.9 |
| amount after 24 hours | | | | |
| (μg/cm²) | | | | |
| Produced amount of | 0.635 | 0.519 | 0.462 | 0.433 |
| menthol ester (%) | | | | |
| Crystal precipitation | none | none | none | none |
| (Initial) | | | | |
| Crystal precipitation | none | none | none | none |
| (3 months) | | | | |
| Crystal precipitation | none | none | none | none |
| (6 months) | | | | |
| Crystal precipitation | none | none | none | none |
| (12 months) | | | | |

TABLE 2

| | Ex. 5 | Ex. 6 | Ex. 7 | Ex. 8 |
|---|---|---|---|---|
| SIS | 20 | 20 | 20 | 20 |
| Alicyclic saturated | 37 | 37 | 34 | 30 |
| hydrocarbon resin | | | | |
| Hydrogenated rosin | — | — | 3 | 7 |
| glycerol ester | | | | |
| Polybutene | 5 | 5 | 5 | 5 |
| Liquid paraffin | 26.05 | 28.1 | 26.08 | 26.08 |
| BHT | 0.3 | 0.3 | 0.3 | 0.3 |
| Ketoprofen | 2 | 2 | 2 | 2 |
| L-menthol | 2.5 | 2.5 | 2.5 | 2.5 |
| Zinc oxide | 0.15 | 0.1 | 0.12 | 0.12 |
| Diisopropyl adipate | 5 | 5 | 5 | 5 |
| Isopropyl myristate | 2 | — | 2 | 2 |
| Ketoprofen:Zinc oxide | 1:0.075 | 1:0.05 | 1:0.06 | 1:0.06 |
| Cumulative permeation | 83.8 | 86.2 | 117.9 | 118.7 |
| amount after 24 hours | | | | |
| (μg/cm²) | | | | |
| Produced amount of | 0.344 | 0.611 | 0.452 | 0.441 |
| menthol ester (%) | | | | |
| Crystal precipitation | none | none | none | none |
| (Initial) | | | | |
| Crystal precipitation | none | none | none | none |
| (3 months) | | | | |
| Crystal precipitation | none | none | none | none |
| (6 months) | | | | |
| Crystal precipitation | none | none | none | none |
| (12 months) | | | | |

TABLE 3

| | Ex. 9 | Com. 1 | Com. 2 | Com. 3 |
|---|---|---|---|---|
| SIS | 20 | 20 | 20 | 20 |
| Alicyclic saturated | 37 | 37 | 37 | 37 |
| hydrocarbon resin | | | | |
| Polybutene | 5 | 5 | 5 | 5 |
| Liquid paraffin | 26 | 26.15 | 26.2 | 33.08 |
| BHT | 0.3 | 0.3 | 0.3 | 0.3 |
| Ketoprofen | 2 | 2 | 2 | 2 |
| L-menthol | 2.5 | 2.5 | 2.5 | 2.5 |
| Zinc oxide | 0.2 | 0.05 | — | 0.12 |
| Diisopropyl adipate | 5 | 5 | 5 | — |
| Isopropyl myristate | 2 | 2 | 2 | — |
| Ketoprofen:Zinc oxide | 1:0.1 | 1:0.025 | — | 1:0.06 |
| Cumulative permeation | 71.1 | 124.8 | 135.4 | 57.7 |
| amount after 24 hours | | | | |
| (μg/cm²) | | | | |
| Produced amount of | 0.222 | 0.822 | 1.127 | 0.357 |
| menthol ester (%) | | | | |
| Crystal precipitation | none | none | none | none |
| (Initial) | | | | |
| Crystal precipitation | none | none | observed | none |
| (3 months) | | | | |
| Crystal precipitation | none | observed | observed | none |
| (6 months ) | | | | |
| Crystal precipitation | none | observed | observed | none |
| (12 months) | | | | |

TABLE 4

| | Com. 4 | Com. 5 |
|---|---|---|
| SIS | 20 | 20 |
| Alicyclic saturated | 37 | 37 |
| hydrocarbon resin | | |
| Polybutene | 5 | 5 |
| Liquid paraffin | 27.9 | 27.7 |
| BHT | 0.3 | 0.3 |
| Ketoprofen | 2 | 2 |
| L-menthol | 2.5 | 2.5 |
| Zinc oxide | 0.3 | 0.5 |
| Diisopropyl adipate | 5 | 5 |
| Isopropyl myristate | — | — |
| Ketoprofen:Zinc oxide | 1:0.15 | 1:0.25 |
| Cumulative permeation | 34.7 | 7.8 |
| amount after 24 hours | | |
| (μg/cm²) | | |
| Produced amount of | 0.008 | 0.002 |
| menthol ester (%) | | |
| Crystal precipitation | none | none |
| (Initial) | | |
| Crystal precipitation | none | none |
| (3 months) | | |
| Crystal precipitation | none | none |
| (6 months) | | |
| Crystal precipitation | none | none |
| (12 months) | | |

TABLE 5

| | Ex. 10 | Ex. 11 | Ex. 12 |
|---|---|---|---|
| SIS | 14 | 30 | 30 |
| Alicyclic saturated | 35 | — | 2 |
| hydrocarbon resin | | | |
| Terpene resin | 5 | 5 | — |
| Hydrogenated rosin | — | 25 | 18 |
| glycerol ester | | | |
| Polybutene | 8 | 2 | 15 |
| Liquid paraffin | 26.08 | 26.08 | 23.08 |
| BHT | 0.3 | 0.3 | 0.3 |
| Ketoprofen | 2 | 2 | 2 |
| L-menthol | 2.5 | 2.5 | 2.5 |
| Zinc oxide | 0.12 | 0.12 | 0.12 |
| Diisopropyl adipate | 5 | 5 | 5 |
| Isopropyl myristate | 2 | 2 | 2 |
| Ketoprofen:Zinc oxide | 1:0.06 | 1:0.06 | 1:0.06 |
| Cumulative permeation | 123.5 | 94.9 | 117.9 |
| amount after 24 hours | | | |
| (μg/cm²) | | | |
| Produced amount of | 0.504 | 0.347 | 0.439 |
| menthol ester (%) | | | |
| Crystal precipitation | none | none | none |
| (Initial) | | | |
| Crystal precipitation | none | none | none |
| (3 months) | | | |

TABLE 6

| | Ex. 13 | Ex. 14 | Ex. 15 |
|---|---|---|---|
| SIS | 12 | 20 | 20 |
| Alicyclic saturated | 37 | 30 | 30 |
| hydrocarbon resin | | | |
| Hydrogenated rosin | 2 | 7 | 7 |
| glycerol ester | | | |
| Polybutene | 12 | 5 | 5 |
| Liquid paraffin | 25.08 | 26.08 | 26.08 |
| BHT | 0.3 | 0.3 | 0.3 |
| Ketoprofen | 2 | 2 | 2 |
| L-menthol | 2.5 | 2.5 | 2.5 |
| Zinc oxide | 0.12 | 0.12 | 0.12 |
| Diisopropyl adipate | 5 | — | 4 |
| Diethyl sebacate | — | 3 | — |
| Isopropyl myristate | 2 | — | — |
| Isopropyl palmitate | — | 4 | 3 |
| Ketoprofen:Zinc oxide | 1:0.06 | 1:0.06 | 1:0.06 |
| Cumulative permeation | 120.2 | 120.0 | 117.5 |
| amount after 24 hours | | | |
| (μg/cm²) | | | |
| Produced amount of | 0.485 | 0.452 | 0.421 |
| menthol ester (%) | | | |
| Crystal precipitation | none | none | none |
| (Initial) | | | |
| Crystal precipitation | none | none | none |
| (3 months ) | | | |

TABLE 7

| | Ex. 16 | Ex. 17 | Ex. 18 |
|---|---|---|---|
| SIS | 20 | 20 | 20 |
| Alicyclic saturated | 30 | 30 | 30 |
| hydrocarbon resin | | | |
| Hydrogenated rosin | 7 | 7 | 7 |
| glycerol ester | | | |
| Polybutene | 5 | 5 | 5 |
| Liquid paraffin | 27.08 | 26.08 | 25.08 |
| BHT | 0.3 | 0.3 | 0.3 |
| Ketoprofen | 2 | 2 | 2 |
| L-menthol | 2.5 | 2.5 | 2.5 |
| Zinc oxide | 0.12 | 0.12 | 0.12 |
| Diisopropyl adipate | — | — | 6 |
| Diethyl sebacate | 3 | 4 | — |
| Isopropyl myristate | — | 3 | — |
| Isopropyl isostearate | 3 | — | 2 |
| Ketoprofen:Zinc oxide | 1:0.06 | 1:0.06 | 1:0.06 |
| Cumulative permeation | 113.3 | 122.3 | 122.0 |
| amount after 24 hours | | | |
| (μg/cm²) | | | |
| Produced amount of | 0.433 | 0.431 | 0.427 |
| menthol ester (%) | | | |
| Crystal precipitation | none | none | none |
| (Initial) | | | |
| Crystal precipitation | none | none | none |
| (3 months) | | | |

### Test Examples

### [Test Example 1] In Vitro Skin Permeability Test

Each patch of Examples 1 to 18 and Comparative Examples 1 to 5 was subjected to an in vitro hairless rat skin permeability test. An excised abdominal skin of a male hairless rat (HWY strain, 7 weeks old) was put in a Franz diffusion cell. The dermis side was set to be the receptor side, the inside of the receptor side was filled with phosphate buffered saline, and hot water of 37° C. was circulated in the water jacket. Each patch was cut into a round shape (1.54 cm 2), and applied to the excised skin. The receptor solution was sampled after the test start until 24 hours, and the amount of the drug permeated the skin was measured by high-performance liquid chromatograph method.

The test results are shown in the above Tables 1 to 7.

### [Test Example 2] Quantitative Test of Menthol Ester

Each patch of Examples 1 to 18 and Comparative Examples 1 to 5 stored under a storage condition of 40° C. for 1 month was cut into a size of 7×10 cm 2, put into a mL centrifuge tube, tetrahydrofuran (hereinafter referred to as “THF”) was added thereto, and the patch was subjected to ultrasonic extraction and extraction with a shaker. The resulting extract was taken into a 50 mL measuring flask, and THF was added thereto such that the volume became 50 mL. A 5 mL of said extract was taken, and a 40% mixed solution of acetonitrile and water was added thereto such that the volume became 50 mL. Then, the resulting solution was filtered through a membrane filter (0.45 μm), and the produced amount of menthol ester of ketoprofen was measure by HPLC method using the following measurement conditions.

The test results are shown in the above Tables 1 to 7. In this regard, the produced amount of menthol ester is shown as the ratio of peak area of each degradation product (%) relative to peak area of the drug ingredient (ketoprofen).

### [HPLC Measurement Conditions]

Column: ACQUITY C18 (2.1×100 mm)
Mobile phase:
A acetonitrile:water:phosphoric acid=100:900:1
B acetonitrile:water:phosphoric acid=900:100:1
Wavelength: 254 nm
Flow rate: 0.4 mL/min
Gradient control

TABLE 8

| Time after | Mobile phase | Mobile phase |
|---|---|---|
| injection (min) | A (%) | B (%) |
| 0 to 10 | 55 → 55 | 45 → 45 |
| 10 to 13 | 55 → 5 | 45 → 95 |
| 13 to 20 | 5 → 5 | 95 → 95 |

As can be understood from the results of Examples 1 to and 9, and Comparative Example 1, and the results of Example 6, and Comparative Examples 4 and 5, the increased amount of zinc oxide suppressed the production of menthol ester, but also decreased the skin permeability of the drug.

Further, as can be understood from the results of Example 2 and Comparative Example 3, the addition of a fatty acid ester in the presence of zinc oxide suppressed the production of menthol ester, and yet showed excellent drug permeability.

### [Test Example 3] Presence or Absence of Crystal Precipitation

Regarding each formulation of Examples 1 to 9 and Comparative Examples 1 to 5 immediately after the production (initial product), and after stored at room temperature for 3 months, 6 months, and 12 months, and each formulation of Examples 10 to 18 immediately after the production (initial product), and after stored at room temperature for 3 months, the presence or absence of crystal precipitation was visually observed.

The test results are shown in the above Tables 1 to 7.

As can be understood from the results in Tables 1 to 7, all formulations did not show crystal precipitation at initial stage. However, Comparative Example 2 which did not comprise zinc oxide showed crystal precipitation after stored for 3 months, and Comparative Example 1 comprising of zinc oxide showed crystal precipitation after stored for 6 months.

Meanwhile, each patch of Examples 1 to 18 of the present invention did not show crystal precipitation after stored for 3 months, and especially each patch of Examples 1 to 9 did not show crystal precipitation after stored for 12 months.

### INDUSTRIAL APPLICABILITY

According to the present invention, a patch which prevents ketoprofen from forming a menthol ester (which can be produced in a formulation during storage or production process), suppresses crystal precipitation even after long storage, and yet shows excellent drug releasability, can be provided.

1. A patch comprising ketoprofen, L-menthol, zinc oxide, and a fatty acid ester in a pasty preparation.

2. The patch according to claim 1, wherein the fatty acid ester is one or more selected from isopropyl myristate, isopropyl palmitate, isopropyl isostearate, diisopropyl adipate, and diethyl sebacate.

3. The patch according to claim 1, wherein the fatty acid ester is one or two selected from isopropyl myristate and diisopropyl adipate.

4. The patch according to claim 1, wherein the fatty acid ester is a combination of a fatty acid diester and a fatty acid monoester.

5. The patch according to claim 1, wherein the contained amount of the ketoprofen is 0.1 to 10% by weight, the contained amount of the L-menthol is 0.1 to 10% by weight, the contained amount of the zinc oxide is 0.03 to 0.5% by weight, and the contained amount of the fatty acid ester is 0.1 to 20% by weight, relative to the pasty preparation weight.

6. The patch according to claim 1, wherein the weight ratio of the contained amount of the ketoprofen and the contained amount of the zinc oxide is 1:0.03 to 1:0.1.

7. The patch according to claim 1 further comprising one or more ingredient(s) selected from a base polymer, a tackifier resin, and a softener in the pasty preparation.

8. The patch according to claim 7, wherein
the base polymer is a styrene-isoprene-styrene block copolymer;
the tackifier resin is one or more selected from a hydrogenated rosin glycerol ester, an alicyclic saturated hydrocarbon resin, and a terpene resin; and
the softener is one or two selected from polybutene and liquid paraffin.

9. The patch according to claim 7, wherein the tackifier resin is one or two selected from a hydrogenated rosin glycerol ester and an alicyclic saturated hydrocarbon resin.

10. The patch according to claim 7, wherein the contained amount of the base polymer is 5 to 50% by weight, the contained amount of the tackifier resin is 10 to 60% by weight, and the contained amount of the softener is 10 to 60% by weight, relative to the pasty preparation weight.

11. The patch according to claim 1 further comprising an antioxidant in the pasty preparation.

12. The patch according to claim 11, wherein the antioxidant is dibutylhydroxytoluene.

13. The patch according to claim 11, wherein the contained amount of the antioxidant is 0.05 to 5% by weight relative to the pasty preparation weight.

14. The patch according to claim 1 for use in the prevention or treatment of inflammation or pain.
